(19) United States
(12) Patent Application Publication
(10) Pub. No.: US 2023/0320772 A1
(43) Pub. Date: Oct. 12, 2023

(54) METHODS AND SYSTEMS FOR COOLING AND HEATING SURGICAL INSTRUMENTS

(71) Applicant: Alexei Babkin, Albuquerque, NM (US)

(72) Inventor: Alexei Babkin, Albuquerque, NM (US)

(21) Appl. No.: 18/021,150

(22) PCT Filed: Aug. 13, 2021
(86) PCT No.: PCT/US2021/045986

§ 371 Date: Feb. 13, 2023

Related U.S. Application Data
(60) Provisional application No. 63/064,922, filed on Aug. 13, 2020.

(51) Int. Cl. A61B 18/02

(52) U.S. Cl. CPC A61B 18/02; A61B 2018/00041

(57) ABSTRACT

A closed refrigerant loop system for fluid communication with a treatment tool that includes a compressor to compress a gas supply of the refrigerant to an elevated pressure; a condenser to cool the compressed gas and convert it to a liquid while at least substantially maintaining the elevated pressure of the refrigerant; and a cryocooler to bring the liquid that is maintained under the elevated pressure to a working temperature for the tip of the treatment tool. The closed refrigerant loop system further comprises a check or expansion valve to receive the refrigerant from the treatment tool; and a warming heat exchanger to provide the gas supply of the refrigerant.

### CROSS-REFERENCE TO RELATED APPLICATIONS

This is a national stage application filed under 35 U.S.C. § 371 based on International Patent Application No. PCT/US2021/045986, filed on Aug. 13, 2021, which claims the benefit of U.S. Provisional Application No. 63/064,922, filed Aug. 13, 2020.

### BACKGROUND

The exemplary embodiments of present invention relate generally to a method and system for providing a refrigerant to a tool useful, for example, for cryoablation treatment.

A probe that is to be used for cryosurgery must be designed with a small shape and size to achieve selective cooling of biological tissues. The cryosurgical system must also be designed to provide reliable cooling (or warming, as desired) of the part of the cryoprobe (i.e., the cryotip) that will be in direct thermal contact with the target biological tissue to be treated.

Many current systems primarily or entirely rely upon expansion. Advantages over evaporation, in which the cooling effect is due to expansion/evaporation of a thermal fluid into its low-density phase with some significant volume change—the present disclosure employs high density cooling fluid, there is no need to manage the low-density gaseous phase. Accordingly, with benefit of the subject disclosure, a cryoprobe can be smaller in diameter. It doesn't depend on the phase change, as an external cooler is involved.

Other current systems employ a single-phase liquid cooling, such as disclosed in U.S. Pat. No. 8,814,850. The presently disclosed subject matter does not rely on a single-phase throughout the system requirement, the gaseous (low-density) phase is allowed outside of the cryoprobe which makes it easier to implement and operate.

Many cryoablation systems (e.g., J-T cooling, evaporation, liquid/critical nitrogen do not provide an efficient warming/thawing mechanism: the warming cycle requires an additional mechanism or uses a gaseous phase which is low-density and hence not efficient. The subject disclosure provides a way to achieve both cooling and warming cycles with similar high efficiency (high density thermal fluid is used in both cases).

### BRIEF SUMMARY OF THE DISCLOSURE

An exemplary embodiment of the subject disclosure provides a closed refrigerant loop system for fluid communication with a treatment tool. The system includes a compressor to compress a gas supply of the refrigerant to an elevated pressure, a condenser to cool the compressed gas and convert it to a liquid while at least substantially maintaining the elevated pressure of the refrigerant, and a cryocooler to bring the liquid that is maintained under the elevated pressure to a working temperature for the tip of the treatment tool. The system further includes a check or expansion valve to receive the refrigerant from the treatment tool, and a warming heat exchanger to provide the gas supply of the refrigerant.

In one embodiment, a check valve receives the refrigerant from the treatment tool. The check valve can be pre-set with a release pressure that ensures a proper pressurized frow of refrigerant to the treatment tool at all times during operation.

In one embodiment, the cryocooler is selected from a thermoelectric cooler (TEC), a liquid nitrogen cooler, a pulse tube refrigerator (PTR), a Stirling cryocooler or a GM cooler. The cryocooler can be in communication with a heat exchanger. In one embodiment, the cryocooler has a capacity of less than 100W or less than 50W.

In one embodiment, the gas supply of the refrigerant to the compressor is at or about ambient temperature and pressure. In one embodiment, the system includes a gas reservoir to receive a flow of refrigerant from the check or expansion valve. In one embodiment, the system further includes a heat exchanger downstream from the check or expansion valve and upstream from the gas reservoir. In one embodiment, the system further includes a liquid reservoir to receive a valved flow of liquid refrigerant from the condenser.

The subject disclosure also provides for warming treatments in which a working fluid is warmed or chilled as desired. Accordingly, another exemplary embodiment of the subject disclosure provides a closed working fluid loop system for fluid communication with a treatment tool. The system includes a compressor to compress a gas supply of the working fluid to an elevated pressure, a condenser to cool the compressed gas and convert it to a liquid while at least substantially maintaining the elevated pressure of the working fluid, and a thermal cycling system. The thermal cycling system includes a cryocooler and an external heater to bring the liquid that is maintained under the elevated pressure to a working temperature for the tip of the treatment tool. The system further includes a check or expansion valve to receive the working fluid from the treatment tool, and a warming heat exchanger to provide the gas supply of the refrigerant.

### BRIEF DESCRIPTION OF THE SEVERAL VIEWS OF THE DRAWINGS

The foregoing summary, as well as the following detailed description of the exemplary embodiments of the subject disclosure, will be better understood when read in conjunction with the appended drawings. For the purpose of illustrating the present disclosure, there are shown in the drawings exemplary embodiments. It should be understood, however, that the subject application is not limited to the precise arrangements and instrumentalities shown.

FIG. 1 is a schematic of a refrigerant loop system according to an exemplary embodiment of the subject disclosure, with identified points A-G;

FIG. 2 is a Pressure (psia) vs. Temperature (° C.) diagram (expected) throughout the loop system described in FIG. 1, including at identified points A-G, based on use of perfluropropane as the working fluid and a desired operating pressure and temperature of about 300 psia and −50° C.; and

FIG. 3 is a schematic of a refrigerant loop system according to an exemplary embodiment of the subject disclosure, with identified points A-G.

### DETAILED DESCRIPTION OF THE DISCLOSURE

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as a non-limiting example.

Throughout the subject application, various aspects thereof can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, the term “cryocooler” is used as commonly understood in the art and refers to a cryogenic cooler for providing active, i.e., external, cooling to cryogenic temperatures and include cryogenic coolers, for example, selected from Stirling coolers, Gifford-McMahon (GM) coolers, pulse-tube coolers, thermoelectric coolers, liquid nitrogen-based coolers, Joule-Thomson cryocoolers, and the like, including functional equivalents to each of foregoing. In certain exemplary embodiments, the cryocooler has a capacity of 100W or less, or 50W or less.

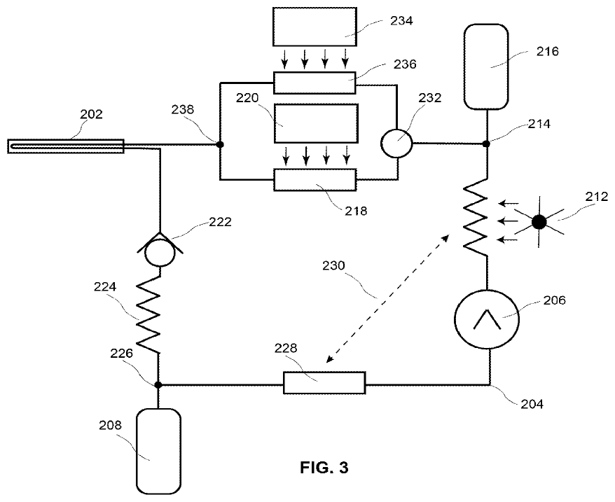

As used herein, the term “refrigerant” and “working fluid” are used interchangeably. More particularly, it is understood that reference to a “refrigerant” would be understood to constitute reference to a “working fluid,” particularly when the fluid is used to provide warming (heat) to a treatment tissue, instead of cooling, as made possible by certain embodiments of the subject disclosure (see FIG. 3).

Furthermore, the described features, advantages, and characteristics of the exemplary embodiments of the subject disclosure may be combined in any suitable manner in one or more embodiments. One skilled in the relevant art will recognize, in light of the description herein, that the subject disclosure can be practiced without one or more of the specific features or advantages of a particular exemplary embodiment. In other instances, additional features and advantages may be recognized in certain embodiments that may not be present in all exemplary embodiments of the present disclosure. Furthermore, description of the corresponding methods of the subject disclosure will be understood from description of the corresponding system.

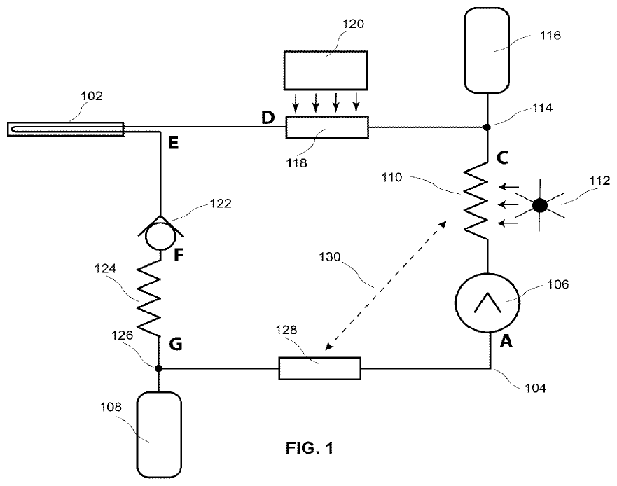

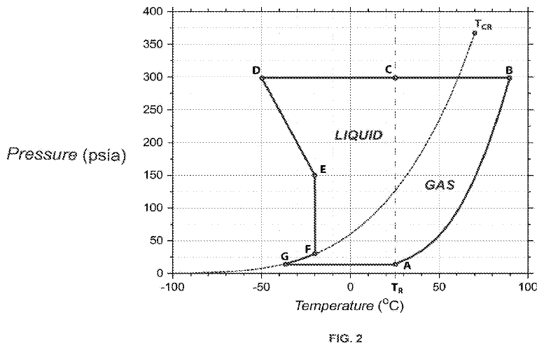

Referring now to the drawings, FIGS. 1 and 2 illustrate a refrigerant loop system 100 for fluid communication with a treatment tool 102 according to an exemplary embodiment of the present application. The treatment tool can be, for example, any surgical instrument for which it is useful to provide a refrigerant, or other working fluid at specific temperatures (e.g., <−45° C.), such as, but not limited to, a cryotip at the distal end of a probe for a cryosurgical procedure). Alternatively, the treatment tool 102 can be, for example and without limitation, a needle, a catheter, a suction cup

In this exemplary embodiment, and as diagrammed in FIG. 2 (expected), the refrigerant or working fluid is perfluropropane, although other working fluids could be used, such as, but not limited to, one or more of propane, nitrogen, argon, nitrous oxide, carbon dioxide, tetrafluoromethane, hexafluoroethane, propane, isobutane, “isopropane” (isobutane/propane mixtures), or other hydrocarbons and fluorocarbons known to those of ordinary skill in the art.

In this closed system, a gas supply 104 of the refrigerant 102 is provided to a compressor 106. The gas supply that provides the source of gas to the compressor can optionally be stored in a gas reservoir 108. In this exemplary embodiment, the gas supply 104 of the refrigerant (i.e., perfluropropane) as it is introduced to the compressor 106 can be at or about ambient or room temperature (T_{R}) and atmospheric pressure, as shown by point A in FIG. 2.

The compressor 106 receives the supply 104 of the refrigerant and compresses the refrigerant in an iso-entropic or substantially iso-entropic manner to an elevated pressure, which in this exemplary embodiment is about 300 psia, which in turn increases the temperature of the gas to about 90° C. (A→C, FIG. 2).

The compressed gas exiting the compressor 106 is then introduced to a heat exchanger or condenser 110 to reduce the temperature of the compressed gas to about T_{R} while maintaining, or substantially maintaining, the elevated pressure, which in this zo exemplary embodiment is about 300 psia (B→C, FIG. 2). This reduction in temperature, while maintaining the elevated pressure, decrease the temperature of the refrigerant to below the boiling point of the refrigerant (T_{CR}) and condenses the gaseous refrigerant to a liquid. A fan 112 is provided in communication with, and/or in close proximity to the condenser 110 to assist in reducing the temperature of the refrigerant.

Refrigerant exiting the condenser 110 in the liquid phase at about ambient temperature and elevated pressure (e.g., about 300 psia) is directed to a valve 114, which can direct a specified flow (e.g., excess capacity) to a liquid reservoir 116.

A heat exchanger 118 is provided downstream of the valve 114, which can receive liquid refrigerant at elevated pressure from the condenser 110 and/or the liquid reservoir 116. The heat exchanger 118 is in communication with a cryocooler 120, which brings the refrigerant to the desired working temperature (C→D, FIG. 2). In this exemplary embodiment, the working temperature is about −50° C., although other working temperatures can be provided depending on the particular medical application of the treatment tool 102. The loop system 100 can be provided with relatively larger tubing and/or low hydraulic resistance from points C→D and from D→treatment tool, 102 (FIG. 1) to minimize any appreciable pressure drop to and from the heat exchanger 118, as the refrigerant flows to the treatment tool 102, in order to ensure proper flow thereto.

In this exemplary embodiment, the cryocooler 102 is a Stirling cooler, although other cryogenic coolers, such as those discussed below, can equally be employed, depending on the application.

Stirling coolers are known in the art and generally include a warm piston, a compression space and warm heat exchanger, a regenerator, a cold heat exchanger, an expansion space, and a cold piston or displacer. A split pair type of Stirling cooler can also find use according to the disclosed subject matter, which generally include two pistons moving in opposite directions driven by AC magnetic fields. Stirling coolers are described, for example, in U.S. Pat. No. 8,196,415, which is hereby incorporated by reference in its entirety.

For cancer treatment applications, in which working temperatures of about −140° C. are typically desired, a 20-40W capacity Stirling cooler or nitrogen-based cryocooler can be employed. Stirling cryocoolers are commercially available from, for example, Stirling Cryogenics (Son, the Netherlands) Sunpower Inc. (Athens, OH), Air Liquide advanced Technologies U.S. LLC (Newark, DE), or other commercial vendors.

Alternatively, the cryocooler can be a nitrogen-based cooler. A source of liquid and/or cooled nitrogen gas (e.g., from a cryogenic storage dewar) can be introduced to heat exchanger 118. Helium-based crycoolers can also find use according to the subject disclosure, which a source of liquid and/or cooled helium is employed to heat exchanger 118.

In certain exemplary embodiments, the cryocooler is a pulse tube refrigerator (PTR), such as a Stirling-type single-orifice PTR. PTR cryocoolers are known in the art. See, e.g., Shaowei, Zhu; Peiyi, Wu; Zhongqi, Chen (1990). “Double inlet pulse tube refrigerators: an important improvement”. Cryogenics. Elsevier BV. 30 (6): 514-520; Matsubara, Y.; Gao, J. L. (1994). “Novel configuration of three-stage pulse tube refrigerator for temperatures below 4 K”. Cryogenics. Elsevier BV. 34 (4): 259-262; Thummes, G.; Wang, C.; Bender, S.; Heiden, C. (1996). Pulsröhrenkühler zur Erzeugung von Temperaturen im Bereich des füssigen Heliums; Xu, M. Y.; De Waele, A. T. A. M.; Ju, Y. L. (1999). “A pulse tube refrigerator below 2 K”. Cryogenics. Elsevier BV. 39 (10): 865-869; Matsubara, Y. (1998). Classification of pulse tube cryocoolers. Proceedings of the 17th International Cryogenic Engineering Conference. Institute of Physics Publishing. pp. 11-16; each of the foregoing being hereby incorporated by reference in their entirety.

In other exemplary embodiments, the cryocooler is a Gifford-McMahon (GM) cooler, which, as is known in the art, generally employs a high pressure line and a low pressure line feed to a water/air cooling compressor, a displacer, a regenerator. In such exemplary embodiments, the compression heat is removed by the cooling water of the compressor via a heat exchanger. The rotary valves alternatingly connect the cooler to the high- and the low-pressure sides of the compressor and runs synchronous with the displacer. GM coolers are described, for example, in U.S. Pat. No. 5,361,588; and Recent Advances in Gifford-McMahon Cryocoolers, R. Vikas and S. Kasthurirengan, 2020, J. Phys.: Conf. Ser. 1473 012052, each of which hereby being incorporated by reference in their entirety.

In other exemplary embodiments, the cryocooler is a thermoelectric cooler (TEC). As is known in the art, a TEC cooler can be connected to a source of DC current that flows through the cooler to create a cold side and a warm side between, for example, alternating p and n-type semiconductor pillars placed thermally in parallel to each other and electrically in series. TEC coolers can particularly find use in skin ablation treatments, which require working temperatures of about −40° C.

The cryocooler 120, in communication with the heat exchanger 118, brings the refrigerant to the working temperature, where it is introduced to, and flowed through, the treatment tool 102. The input of working fluid to the treatment device is always in a liquid and high-density state. Contact with the treatment tissue will increase the temperature of the refrigerant due to heat exchange with the treatment tissue, and flow of the liquid refrigerant through the treatment tool also reduces the pressure of the refrigerant (D→E, FIG. 2).

The “spent” liquid refrigerant is introduced to a check valve 122, which expands the refrigerant in an iso-enthalpic manner. The reduction in pressure, in this exemplary embodiment from about 150 psia to about 30 psia, brings the refrigerant at or near the boiling point of the refrigerant (E→F, FIG. 2) . Appropriate setting of the release pressure for the check valve 122 ensures that the refrigerant remains in a high-density state inside the cryoprobe throughout operation, with sufficient operation pressure at all times. Alternatively, an expansion valve can be used in place of the check valve 122, although a check valve is preferred for the reasons sets forth above.

The expanded gas is then introduced to a heat exchanger 124 where it is cooled and further reduced to ambient pressure, thereby converting the refrigerant to a gas (F→G, FIG. 2).

The converted gas is then introduced to a valve 126, which can direct the gas refrigerant to the gas reservoir 108, and/or to warming heat exchanger 128 that warms the gas to ambient temperature (G→A, FIG. 2). The refrigerant, now substantially at ambient temperature and pressure provides the gas supply 104 to the compressor 106, thereby completing the closed loop. In this exemplary embodiment, condenser 110 is in thermal communication with warming heat exchanger 128, receiving heat from condenser 110 via heat pipe 130. In other embodiments, the heat pipe 130 is not present and the warming heat exchanger 128 is not thermally linked with the condenser 110.

FIG. 3 illustrate a refrigerant loop system 200 for fluid communication with a treatment tool 102 according to an alternative, exemplary embodiment of the present application. Refrigerant loop system 200 is similar to refrigerant loop system 100, and similarly includes a compressor 206 to compress a gas supply 204 of the refrigerant to an elevated pressure, a condenser 210, a check valve 222 to receive the refrigerant from the treatment tool; a heat exchanger 224 downstream of the check valve 222, and a warming heat exchanger 228 to provide the gas supply of the refrigerant.

Also, similar to refrigerant loop system 100, the refrigerant loop system 200 includes a fan 212 in proximity to the condenser 210, which in conjunction with heat pipe 230, provides a thermal link to warming heat exchanger 228 in this particular exemplary embodiment. In other embodiments, the heat exchangers are not thermally linked. And as with refrigerant loop system 100, the refrigerant loop system 200 similarly includes, optionally, gas reservoir 208 and liquid reservoir 216, which in conjunction, respectively, with valves 226 and 214, allow the system to incorporate a higher volume of refrigerant to ensure adequate flows of refrigerant at all times and easier operation. In alternative embodiments, liquid and/or gas reservoirs are not included. In any event, the input of working fluid to the treatment device is always in a liquid and high-density state.

In this exemplary embodiment, the refrigerant loop system 200 includes a three way valve 232, an external heater 234, in addition to an cryocooler 220 which collectively provide thermal cycling to allow a refrigerant (i.e., a working fluid) to be chilled and warmed such that tissues can likewise be chilled or warmed, or subjected to periodic cycles of each upon application of the treatment tool 102. As shown in FIG. 3, the three-way valve 232 is located downstream from the valve 214, which in turn receives working fluid compressed by compressor 206 and condensed to a liquid by condenser 210. The external heater 234 is provided in communication with a heat exchanger 236, and the cryocooler is provided in connection with a heat exchanger 218. A second three-way valve 238 is provided downstream of the external heater 234 and cryocooler 220 to meter flow to the treatment tool 102.

In certain embodiments, the refrigerant loop system consists only of a single loop of refrigerant or working fluid, as opposed to multiple flow paths of primary and secondary working fluid.

It will be appreciated by those skilled in the art that changes could be made to the exemplary embodiments described above without departing from the broad inventive concept thereof. It is to be understood, therefore, that this disclosure is not limited to the particular embodiments disclosed, but it is intended to cover modifications within the spirit and scope of the subject disclosure as disclosed above.

1. A closed refrigerant loop system for fluid communication with a treatment tool comprising:
(a) a compressor to compress a gas supply of a refrigerant to an elevated pressure;
(b) a condenser to cool the compressed gas and convert the compressed gas to a liquid while at least substantially maintaining the elevated pressure of the refrigerant;
(c) a cryocooler to bring the liquid that is maintained under the elevated pressure to a working temperature for a tip of the treatment tool;
(d) a check or expansion valve to receive the refrigerant from the treatment tool; and
(e) a warming heat exchanger to provide the gas supply of the refrigerant.

2. The closed refrigerant loop system of claim 1, wherein a check valve receives the refrigerant from the treatment tool in step (d).

3. The closed refrigerant loop system of claim 1, wherein the cryocooler is selected from a thermoelectric cooler (TEC), a liquid nitrogen cooler, a pulse tube refrigerator (PTR), a Stirling cryocooler or a GM cooler.

4. The closed refrigerant loop system of claim 1, wherein the cryocooler is in communication with a heat exchanger.

5. The closed refrigerant loop system of claim 1, wherein the gas supply of the refrigerant to the compressor is at or about ambient temperature and pressure.

6. The closed refrigerant loop system of claim 1, further comprising a gas reservoir to receive a flow of refrigerant from the check or expansion valve.

7. The closed refrigerant loop system of claim 6, further comprising a heat exchanger downstream from the check or expansion valve and upstream from the gas reservoir.

8. The closed refrigerant loop system of claim 1, further comprising a liquid reservoir to receive a valved flow of liquid refrigerant from the condenser.

9. The closed refrigerant loop system of claim 1, wherein the cryocooler has a capacity of less than 100W.

10. The closed refrigerant loop system of claim 9, wherein the cryocooler has a capacity of less than 50W.

11. A closed working fluid loop system for fluid communication with a treatment tool comprising:
(a) a compressor to compress a gas supply of a working fluid to an elevated pressure;
(b) a condenser to cool the compressed gas and convert it to a liquid while at least substantially maintaining the elevated pressure of the working fluid;
(c) a thermal cycling system comprising a cryocooler and an external heater to bring the liquid that is maintained under the elevated pressure to a working temperature for a tip of the treatment tool;
(d) a check or expansion valve to receive the working fluid from the treatment tool; and
(e) a warming heat exchanger to provide the gas supply of the working fluid.

12. The closed working fluid loop system of claim 11, wherein a check valve receives the working fluid from the treatment tool in step (d).

13. The closed working fluid loop system of claim 11, wherein the cryocooler is selected from a thermoelectric cooler (TEC), a liquid nitrogen cooler, a pulse tube refrigerator (PTR), a Stirling cryocooler or a GM cooler.

14. The closed working fluid loop system of claim 11, wherein the cryocooler has a capacity of less than 100W.

15. The closed working fluid loop system of claim 14, wherein the cryocooler has a capacity of less than 50W.
